(19) United States
(12) Patent Application Publication
(10) Pub. No.: US 2005/0113709 A1
(43) Pub. Date: May 26, 2005

(54) Diagnostic system and methods for detecting disorders in the respiratory control chemoreceptors

(75) Inventor: Yoav Millet, Franklin Lakes, NJ (US)

Correspondence Address: Martin MOYNIHAN;c/o ANTHONY CASTORINA, SUITE 207, 2001 JEFFERSON DAVIS HIGHWAY, ARLINGTON, VA, 22202, US

(73) Assignee: Titanium Ventures Inc.

(21) Appl. No.: 10/946,013

(22) Filed: Sep. 22, 2004

Related U.S. Application Data
(60) Provisional application No. 60/524,403, filed on Nov. 24, 2003.

(51) Int. Cl. A61B 5/02; A61B 5/08

(52) U.S. Cl. 600/529

(57) ABSTRACT

A diagnostic system and methods for detecting abnormalities in central and peripheral respiratory control chemoreceptors is provided. The system and methods are based on distinguishing between relative sensitivities of chemoreceptors of individuals with normal and compromised chemoreceptors. Predetermined combined carbon dioxide and oxygen levels are provided to a subject for a specified period of time, during which physiological parameters are measured as an indication of response to the chemoreceptors relative sensitivities. A preferred method provides higher than normal combined concentrations of carbon dioxide and oxygen, and lower than normal combined concentrations of carbon dioxide and oxygen, and measures physiological parameters to calculate an index, taking into account the basic parameters of the patient such as age, cardio-pulmonological condition and BMI, and provide a diagnosis regarding the function of the respiratory control chemoreceptors.

### RELATED PATENT APPLICATION

This application claims priority from U.S. Provisional Patent Application No. 60/524,403, filed on Nov. 24, 2003.

### FIELD AND BACKGROUND OF THE INVENTION

The present invention relates to a diagnostic system and methods for detecting adaptive related disorders of the respiratory control chemoreceptors (hereinafter “RCC”) and, more particularly, to a system and methods for diagnosing RCC abnormalities based on the relative and combined sensitivities of CO₂ and O₂ chemoreceptors and an individual's adaptation thereto.

The diagnosis of RCC that have undergone adaptive changes could be useful in detecting potential various underlying health conditions, including sleep apnea related disorders and SIDS.

Sleep Apnea Syndrome

Sleep apnea syndrome (SAS) is a breathing disorder characterized by apneas (cessation of airflow for ten seconds or more) and hypopneas (a decrease in flow by at least 50% for 10 seconds or more). Both apneas and hypopneas are associated with sleep arousal and/or oxygen desaturations of 3% or more. Apneas and, hypopneas result from upper airway occlusion, either full or partial, or from a loss of the autonomic drive to breathe.

There are three types of apnea: obstructive, central, and mixed. Obstructive sleep apnea. (OSA) is the most common type of sleep apnea. OSA occurs when the upper airway occludes (either partially or fully) but efforts to breathe continue. The primary causes of upper airway obstruction are lack of muscle tone during sleep, excess tissue in the upper airway, and anatomic abnormalities in the upper airway and jaw. Central sleep apnea (CSA) affects only 5-10% of the sleep apnea population. CSA occurs when both airflow and respiratory effort cease. This cessation of breathing results from a loss of the autonomic drive to breathe. Mixed apnea occurs when an initial central component followed by an obstructive component causes a cessation of breathing.

In all three types of apnea, breathing resumes when the patient has a brief arousal from sleep, of which they usually have no memory. In severe cases, patients may have up to 100 events per hour, resulting in severe daytime symptomatology. Disease severity is usually classified according to the apnea/hypopnea index (AHI). Measured during a sleep study, AHI refers to the number of apneas and hypopneas per hour. An AHI of 5 or more generally indicates the presence of mild SAS, and an AHI of 15 generally indicates moderate SAS.

Polysomnography (PSG) is used to evaluate abnormalities of sleep and/or wakefulness and other physiologic disorders that have an impact on or are related to sleep and/or wakefulness. A polysomnogram is used to diagnose and treat sleep apnea symptoms (SAS), and consists of simultaneous recording of multiple physiologic parameters related to sleep and wakefulness. The interaction of various organ systems during sleep and wakefulness also is evaluated.

A polysomnogram has several neurophysiologic channels, including at least one electroencephalography (EEG) channel to monitor the sleep stage, two electrooculogram (EOG) channels to monitor horizontal and vertical eye movements, and one electromyography (EMG) channel to record atonia of rapid eye movement (REM) sleep. Other parameters often monitored include additional EEG channels, particularly in patients with sleep-related epilepsy, additional EMG channels, particularly the anterior tibialis, to detect periodic limb movements of sleep, airflow, electrocardiogram (ECG), pulse oximetry, respiratory effort and sound recordings to measure snoring. In addition, several other parameters are also optionally monitored, including video monitoring of body positions, core body temperature, incident light intensity, penile tumescence, pressure and pH at various esophageal levels, CO₂ capnograph with correlation to apnea, and abdominal and chest plathysmographs to monitor breathing effort.

Sleep study laboratories, which use polysomnography, are generally expensive and not widely available for a large segment of the population. According to several publications, only about 5% of sleep disorder patients have been diagnosed for this condition. Sleep arousals related to obstructive events are associated with increases in blood pressure and heart rate. Chronic hemodynamic consequences of SAS include hypertension, diurnal hypertension, and pulmonary hypertension. Additionally, some evidence indicates that left ventricular mass is greater in patients with SAS than in age-matched controls. One study demonstrated an increase in hypertension in patients with an AHI greater than 5 events per hour as compared to a control group matched for obesity, age, and gender. Studies have also demonstrated increased sympathetic nerve activity in patients with OSA, and researchers suggest that this mechanism contributes to the development of hypertension in SAS patients.

SIDS (Sudden Infant Death Syndrome)

Another RCC disorder for which pre-screening could be advantageous is sudden infant death syndrome (SIDS). SIDS is the unexpected and sudden death of an apparently healthy infant during sleep. It is the leading cause of death in infants between two weeks and one year of age, striking about one per 1,000 infants. Since SIDS generally strikes without warning, it would be helpful to have a system and method for identifying infants at risk and applying special monitoring and therapy techniques for such infants.

Thus, it would be highly advantageous to have an inexpensive, widely available system and method for pre-screening and early detection of adaptive RCC disorders.

### SUMMARY OF THE INVENTION

According to one aspect of the present invention there is provided a method of diagnosing RCC abnormalities, including determining in a subject a physiological response to predetermined carbon dioxide levels so as to obtain a first value, determining in a subject a physiological response to predetermined oxygen levels so as to obtain a second value, combining the first and second values for a differential comparison, and diagnosing an RCC disorder in the subject based on the comparison.

According to antoher aspect of the present invention there is provided a method of diagnosing a sleep disorder, including determining in a subject at least one physiological response to one or several predetermined combined carbon dioxide and oxygen levels in a breathing gas mixture, and diagnosing a sleep disorder in the subject based on the determined physiological response. The physiological response can include the measurement of the respiratory volume (VE), arterial oxygen saturation, breathing rate, sympathetic nerve activity, heart rate, pulse, blood flow, bicarbonate levels, or any other suitable parameter or combination thereof.

In different embodiments, the carbon dioxide and oxygen levels are administered to the subject in normal air composition amounts, higher than normal concentrations of both, or lower than normal concentrations of both. The determining can be done over a period of 10-60 seconds, or over a period of 1-20 minutes. The RCC abnormalities can indicate potential breathing disorders such as sleep apnea, SIDS, cardio-pulmonology disorder or any other relevant disorder.

According to another aspect, of the present invention, there is provided a method for providing a diagnostic measure of RCC function, including providing to a subject a predetermined ratio of oxygen and carbon dioxide, measuring a breathing parameter of the subject in response to the provided combination, comparing the breathing to a baseline parameter, and calculating an index based on the comparison, the index serving as the diagnostic measure. The index may be reported via cable or wireless communication to a health care provider for diagnosis.

According to yet another aspect of the present invention, there is provided a system for diagnosing RCC f-unction, including a device for determining in a subject at least one physiological response to predetermined carbon dioxide and oxygen levels, and a processor for diagnosing the RCC disorder based on the physiological breathing response. In a preferred embodiment, the device includes a gas mixer having therein a gas mixture of a predetermined ratio of oxygen and carbon dioxide, a gas introducer connected to the breathing gas mixer for introducing the gas mixture to a user, and a sensor connected to the user and the processor for measurement of at least one breathing response parameter. The processor includes a gas regulator in communication with the breathing gas mixer, an input data collector in communication with the sensor, and an index calculator in communication with the input data collector. The system may further include a display in communication with the processor for display of the calculated index.

According to yet another aspect of the present invention, there is provided a system for providing a diagnostic measure of RCC function, including a breathing gas combination provider, and a breathing parameter measurer for measuring at least one breathing response of a user in response to breathing the breathing gas combination. The system also includes a comparator for comparing the measurement to a predetermined baseline parameter, and an evaluator for providing a diagnosis based on the comparison.

The present invention successfully addresses the shortcomings of the presently known configurations by providing a method for pre-screening and early detection of breathing disorders related to RCC desensitization, including sleep apnea and SIDS.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a graphical illustration of a comparison of normal and compromised chemoreceptor responses to varying amounts of carbon dioxide;

FIG. 2 is a graphical illustration of a comparison of normal and compromised chemoreceptor responses to varying amounts of oxygen;

FIG. 3 is a flow chart illustration of an overview of the steps of a method for pre-screening for a sleep disorder;

FIG. 4 is an illustration of a system in accordance with a preferred embodiment of the present invention;

FIG. 5 is a block diagram illustration of a processor of the system of FIG. 4 in greater detail;

FIG. 6 is a flow chart illustration of a method of using the system of FIGS. 4 and 5; and

FIG. 7 is a graphical illustration of expected results useful in providing a diagnosis in accordance with a preferred embodiment of the present invention.

### DESCRIPTION OF THE PREFERRED EMBODIMENTS

The present invention is of a diagnostic system and methods for detecting RCC disorders. Specifically, the present invention can be used to detect an RCC disorder by measuring responses to predetermined and combined ratios of O₂ and CO₂. The invention described herein can provide pre-screening for patients, who can then be monitored, treated or referred for further examination. More particularly, the present invention is of a diagnostic system and method which can be used for screening for a breathing disorder, such as sleep apnea or SIDS.

Normal breathing is a complex physiological process primarily involving inhalation of oxygen enriched air and exhalation of carbon dioxide enriched air. On a basic level, when oxygen enters the lungs, it diffuses from the alveoli into the blood stream. Carbon dioxide bound to hemoglobin in the blood is released, and the hemoglobin binds with the oxygen. The released carbon dioxide diffuses from the blood to the alveoli, and is exhaled.

Breathing is regulated by a complex interaction of physiological processes. An important role in regulation is played by chemoreceptors, which are specialized nerves that detect imbalances in blood gases (O₂ and CO₂) and in pH. Carbon dioxide and oxygen levels in the blood are regulated by peripheral chemoreceptors. However, carbon dioxide is able to cross the blood brain barrier and is therefore measurable in the extracelluiar fluid (ECF) as well. Central chemoreceptors, located in the medulla, regulate carbon dioxide levels in the ECF. Both peripheral and central chemoreceptors are sensitive to pH and signal the respiratory centers in the brain if either the blood or the ECF pH (i.e. concentration of H⁺ ions) changes beyond predetermined levels.

H⁺ is continually produced in the body as a by-product of metabolism (lactic acid, CO₂, etc.) and is maintained in a narrow physiologic range. Normal pH in the ECF is 7.4, as shown in the following equation:
pH=−log[_{H}^{+]=-log(}10^{-7.4})=7.4

The body maintains this narrow ECF physiologic pH by chemical and biochemical buffers, which react quickly to compensate for addition or subtraction of H⁺ from the body. Such buffers include HCO₃⁻, and Hb⁻, among others. HCO₃⁻ in particular is abundant, measurable, and can be compensated for by the respiratory and urinary systems. The Henderson-Hasselbach equation demonstrates a relationship between the pH and the ratio of HCO₃⁻ to H₂CO₃, as follows:

$$pH = pK + log \frac{{HCO}_{3}^{-}}{{H}_{2} {CO}_{3}}$$

Thus, an increase or decrease in HCO₃⁻ can compensate for changes in pH. Furthermore, changes in pH can be compensated for by the respiratory system, by controlling CO₂ elimination. The respiratory system takes 1-3 minutes to compensate for changes in pH. Lastly, pH can be compensated for by the urinary system, by control of elimination of HCO₃⁻ via the kidneys. It takes several hours to days for the urinary system to compensate for changes in pH.

Thus, there is a delicate balance between CO₂ and HCO₃⁻, and their interplay with one another in maintaining pH, as can be seen in the following equation:
CO₂+H₂O [symbol] H⁺+HCO₃⁻

During normal breathing, CO₂ easily diffuses into the CSF (cerebrospinal fluid), causing a decrease in the pH which signals the central chemoreceptors in the brain which, in turn, increase the breathing rate, usually measured in breaths per minute (BPM) or respiratory volume of air (VE). Being a charged molecule, bicarbonate does not normally cross the blood brain barrier. However, in chronic hypercapnea (greater than 6 weeks duration), the body will begin actively transporting bicarbonate into the CSF to compensate for the increased CO₂. This increases the pH around the CO₂ chemoreceptors, which causes them to cease signaling under normal CO₂ levels.

Chronic hypercapnea, which is one of the mechanisms which leads to an adaptive shift in the relative sensitivity of the RCC such as the one described above, may be caused by certain breathing disorders, such as OSA (obstructive sleep apnea) or by other underlying disorders which can cause a tendency towards SIDS, or other cardiopulmonary or respiratory disorders in which the sensitivity of an individual becomes abnormal with respect to levels of carbon dioxide in the ECF. This type of adaptation can lead to further deterioration of the respiratory system, wherein the individual is not fully aware of the lack of oxygen because the body does not respond to the normal indications that breathing should take place. Thus, conditions such as central sleep apnea (CSA) can develop, or in infants, the condition can lead to SIDS. Similar symptoms have also been shown in deep divers chronically exposed to elevated CO₂ levels. This group of individuals can collectively be referred to as CO₂ retainers.

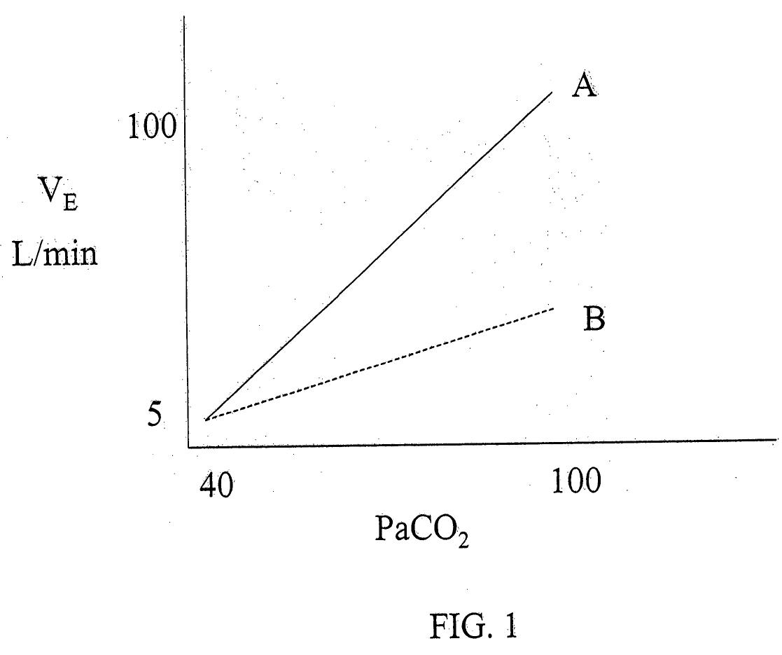

Reference is now made to FIG. 1, which is a graphical illustration of a comparison of normal and compromised chemoreceptor responses to varying amounts of carbon dioxide. As shown on the X-axis, the partial pressure of carbon dioxide is varied from 40 to 100 percent, and responses are measured as VE in Liters per minute. As shown in FIG. 1, as the artial pressure of carbon dioxide (PaCO₂) is increased, breathing increases linearly. However, the total VE as well as the slope of the curve is lower in individuals with compromised chemoreceptors, as denoted in line B, than it is in normal individuals, as denoted in line A. Thus, as an individual with compromised chemoreceptors is given higher amounts of CO₂, the chemoreceptors do not detect the increase as readily or as rapidly as in normal individuals.

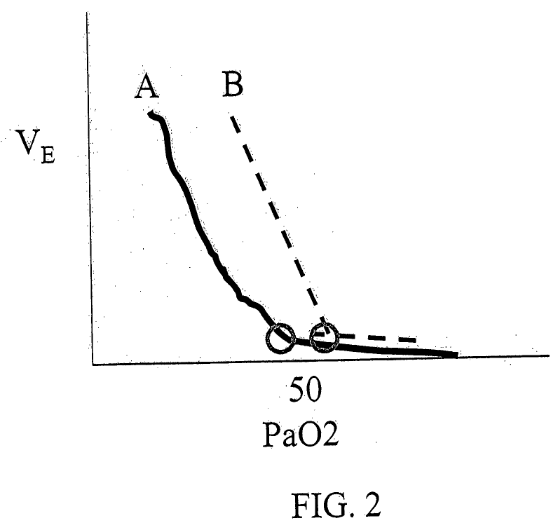

Reaction curves to PaO₂ are generally non-linear, with a decrease in VE occurring in response to an increase in oxygen. An example of this type of curve is shown in FIG. 2. Reactions to a decrease in oxygen in normal individuals, as shown in line A, results in a decrease in breathing, wherein the amount of breathing approaches zero at a PaO₂ of approximately 50. However, in individuals with compromised chemoreceptors, as shown in line B, the saturation point is not reached until oxygen levels are present in higher numbers, such as a PaO₂ of 60 or 70. Thus, possibly as compensation for the compromised CO₂ receptors, research has shown that OSA patients have significantly higher sensitivity to oxygen levels than non-OSA controls. For example, according to Smith et al. in “Effects of hypoxia on sympathetic neural control in humans,” in Respiration Physiology 121 (2000) 163-171, OSA patients reacted to O₂ saturation levels of 82-90% compared to a below 80% saturation reaction level of healthy individuals with the same BMI and age. The measured reaction was increased BPM and SNA (sympathetic nerve activity). Although, the reason for this elevated oxygen sensitivity was stated in this research as unknown, this phenomenon may be a direct consequence of the compromised CO₂, chemoreceptors, which are compensated for by peripheral chemoreceptor hypersensitivity to oxygen levels in order to maintain respiratory regulation. Since O₂ receptors are peripheral, they are not affected by the bicarbonate level and the ECF pH.

Both the decreased sensitivity of CO₂ chemoreceptors and increased sensitivity Of O₂ receptors can be used to distinguish those with RCC disorders such as OSA or SIDS from the rest of the population, particularly when used in combination with one another.

It is an object of the present invention to provide methods for pre-screening and diagnosing of RCC disorders based on the above principles. Patients who are suspected of having an RCC disorder would be screened using the methods of the invention and could then be referred for further testing, monitoring and treatment when necessary.

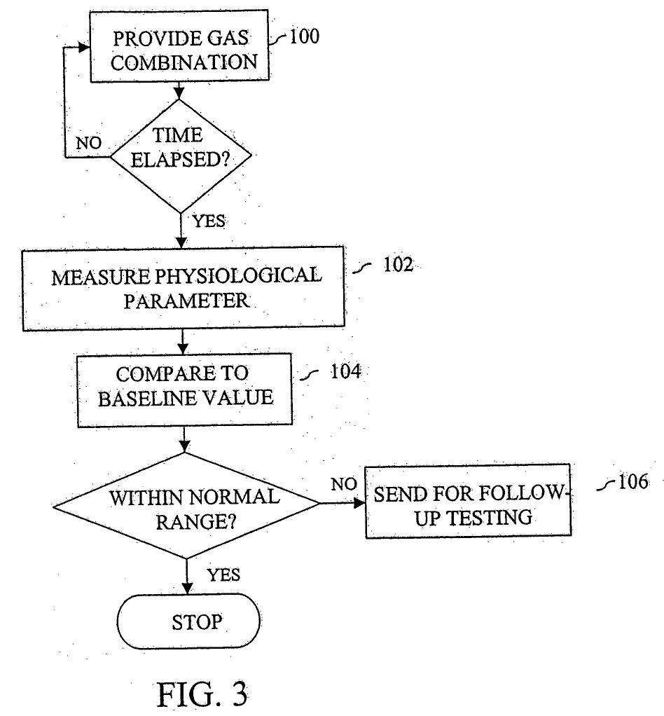

Methods according to the present invention may be better understood with reference to FIGS. 3-7 and their accompanying descriptions. The methods described herein are suitable for people with, normal cardio-pulmonology function. Patients with lung or cardiology conditions which might compromise their breathing patterns in a way that would interfere with the testing procedures would be excluded or tested with adjusted baseline if applicable. An initial spirometry test is included as an optional first step prior to testing.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Reference is now made to FIG. 3, which is a flow chart illustration of an overview of the steps of a method for pre-screening for an RCC disorder, in accordance with a preferred embodiment of the present invention. Initially, an administrator provides (step 100) a gas combination to the subject. Several combinations may be introduced individually. In one embodiment, the gas combination is normal air. In another embodiment, the gas combination is adjusted air, wherein combined higher or lower ratios of oxygen and/or carbon dioxide are used. The gas combination is administered to a subject at rest or during physical exertion, such as a stress test, depending on the type of test to be performed, as described in further detail hereinbelow. The subject inhales the gas combination for a predetermined amount of time. In one embodiment, the duration for inhalation is 2-20 minutes. In a preferred embodiment, the duration for inhalation is approximately 5 minutes. During the inhalation period, a particular physiological parameter is measured (step 102). The physiological parameter varies with the type of test and may include any one of or combination of: volume of air inhaled or exhaled, amount of exhaled CO₂, amount of saturated O₂ (via pulse oximeter or any other suitable method), breathing rate, pulse and sympathetic nerve activity. The measured parameter is then compared (step 104) to a baseline value for normal individuals. If the measurement is in the normal range, the test is ended. Otherwise, the subject is referred for further testing and treatment.

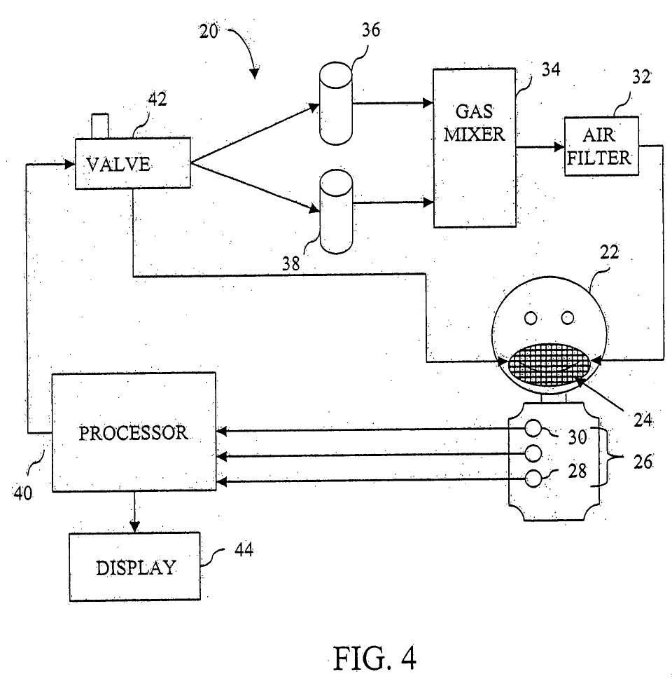

Reference is now made to FIG. 4, which is an illustration of system 20 in use with a subject. A subject 22 is equipped with a breathing mask 24 and a group of sensors, generally referred to as 26. The group of sensors 26 includes a regulating sensor 28 for regulating gas pressures, a physiological sensor 30 for detecting a physiological parameter in response to breathing, and optionally additional sensors for monitoring additional vital signs such as heart rate and blood pressure. Breathing mask 24 is connected through an air filter 32 to a gas mixer 34. Gas mixer 34 receives an adjustable mixture of gases from a first balloon 36 and a second balloon 38. Sensors 26 are connected to a processor 40. Processor 40 is further connected to a valve 42, which allows direct administration of room air to mask 24, or administration of any combination of first and second balloons 36 and 38 through gas mixer 34 and air filter 32.

Devices for CPAP (continuous positive airway pressure) are known in art. Such devices may be modified for the present application. In a preferred embodiment, breathing mask 24 is any commercially available mask, such as the Mirage™ series from ResMed Corp., Poway, Calif., USA. In an alternative embodiment, breathing mask 24 is an enclosed chamber, which can be fitted over the entire head. Breathing mask 24 can be connected to a gas mixer 34, such as the type found in a CPAP device, for example, the S6™ or S7™ from ResMed Corp. Sensors 26 can include any suitable types of sensors. In one embodiment, regulating sensor 28 and physiological sensor 30 are the same sensor, so that only one sensor is used to both provide feedback to processor 40 and to provide physiological information to be used in the diagnosis. In a preferred embodiment, regulating sensor 28 is a pressure sensor. For example, regulating sensor 28 may be an airflow sensor, such as the Pro-Tech® Respiratory Airflow Sensor from Repironics, Inc., Pittsburgh, Pa., USA. In an alternative embodiment, regulating sensor 28 is a chemical sensor, for analyzing gas content. Regulating sensor can-be attached to breathing mask 24, or can be located on the body of the subject in a separate location. In one embodiment, physiological sensor 30 is a pressure sensor, which can be used to detect breathing rate (breaths per minute). In another embodiment, physiological sensor 30 is a pulse oximeter, such as the 920M™ Plus Pulse Oximeter from Respironics, Inc., Pittsburgh, Pa., USA, which can be used to detect the amount of hemoglobin saturated with oxygen.

First balloon 36 and second balloon 38 may contain any suitable combination of oxygen and carbon dioxide, along with appropriate amounts of nitrogen and possibly some of the other gases normally found in air. In a preferred embodiment, first balloon 36 has a low percentage of oxygen and a low percentage of carbon dioxide, while second balloon 38 has a high percentage of oxygen and a high percentage of carbon dioxide. In another embodiment, additional balloons are included, having low percentages of carbon dioxide together with high percentages of oxygen and vice versa. Normal air includes several gases, at the following approximate percentages: 78% Nitrogen; 21% Oxygen; 0.03% Carbon Dioxide; 0.9% Argon; and trace amounts of Neon, Methane, Helium, Krypton, Hydrogen and Xenon. Thus, high percentages of oxygen are in a range of 30-100%, while low percentages of oxygen include are in a range of 5-15%. High percentages of carbon dioxide are in a range of 1-10%, while low percentages of carbon dioxide are less than 0.02%. Any combination of types of air is possible, including variable percentages of carbon dioxide and oxygen.

Processor 40 controls the gas content, and can variably adjust it by allowing each balloon to open or close more or less. Processor 40 also controls valve 42, which allows breathing mask 24 to be closed to air from balloons 36 and 38 and open to normal air, or vice versa. Additionally, processor 40 collects data from sensors 26, both relating to regulation of air flow and relating to measured responses to the air flow. This type of loop allows for, adjustments during the testing procedure, and termination of the test if any danger is indicated.

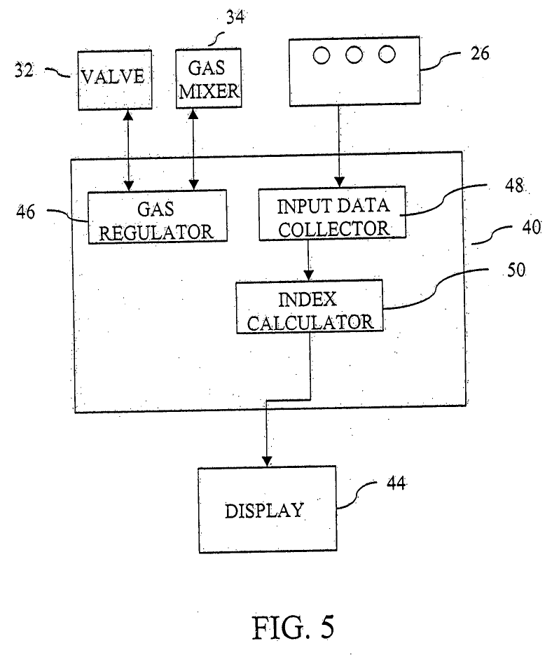

Reference is now made to FIG. 5, which is a block diagram illustration of processor 40 in greater detail. Processor 40 includes a gas regulator 46 in communication with gas mixer 34 and valve 32, an input data collector 48 in communication with gas regulator 46 and with sensors 26, and an index calculator 50 in communication with input data collector 48 and display 44. Gas regulator 46 receives data from input data collector 48 regarding breathing rate and gas composition, and provides adjustments to gas mixer 34 and/or valve 32 based on the data. Thus, gas regulator 46 controls the composition of gases provided to the subject via breathing mask 24, and is configured to signal the valve to open or close, fully or partially, so as to allow for the introduction of normal air or not. Input data collector 48 receives data from sensors 26, relating to breathing rate and gas composition, as well as measurable physiological parameters. Index calculator 50 calculates an index to be used for diagnosis based on comparison of the data received from input data collector 48 with known normal values. The calculated indices are sent to display 44, either via a cable or a wireless connection. In a preferred embodiment, display 44 is a report which is sent to a practice or hospital database, which can be used by a physician for diagnosis. In alternative embodiments, display 44 is a graph or a chart.

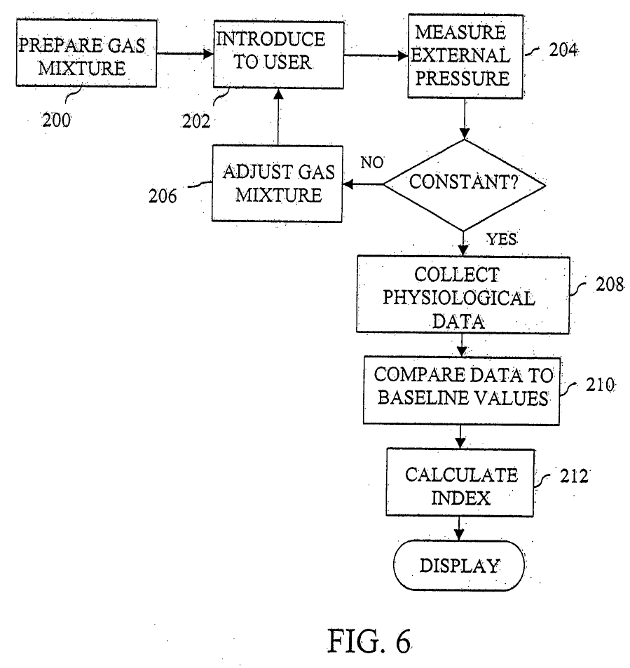

Reference is now made to FIG. 6, which is a flow chart illustration of a method of using system 20, according to a preferred embodiment of the present invention. Initially, gas mixer 34, based on input from processor 40, and via breathing mask 24, provides (step 200) a suitable mixture of gases to be administered (step 202) to the user. Gas mixer 34 is in communication with gas regulator 42, for regulating and adjusting the individual partial pressures of the gases within gas mixer 34. Gas regulator 42 can be a part of processor 40 or a separate unit. Optionally, a regulating sensor 28 is located on or in the breathing mask or chamber, and is configured to measure (step 204) relative external pressure before and during each breathing phase. If the external pressure changes, sensor 26 signals gas regulator 42 to adjust (step 206) the partial pressures of the gases within gas mixer 34. Regulating sensor 28 may alternatively be a chemical sensor to measure the composition of gas being provided and adjusting it accordingly. Physiological sensor 30 is configured to detect a physiological parameter, examples of which are provided hereinbelow. Input data collector 48 collects (step 208) data from physiological sensor 30, and sends the data to index calculator 50. Index calculator 50 compares (step 210) the received data to baseline values, and calculates (step 212) an index based on the comparison. Processor 40 then sends the final data to display 44.

There are several possibilities for measurements of physiological parameters. In a preferred embodiment, the measured parameter is VE. VE may be measured using any commercially available CPAP, such as the ResMed S6™ or S7™ series. In an alternative embodiment, the measured parameter is arterial saturation with hemoglobin (SaO₂). At rest, expected VE or SaO₂ of RCC disordered breathing (RDB) individuals exposed to normal air is approximately equal to that of normal individuals. With exercise, expected VE or SaO₂ of RCC disordered breathing (RDB) individuals exposed to normal air would decrease, while that of normal individuals would remain relatively stable due to the increased respiration brought on by elevated CO₂ levels. In an alternative embodiment, the measured parameter is breathing rate, measured by a pressure sensor in breaths per minute. Normal values for SaO₂ at rest are 90-100%, and normal values for breathing rate are 10-14 breaths per minute. Deviations from these values (+/−2%) would indicate an abnormality.

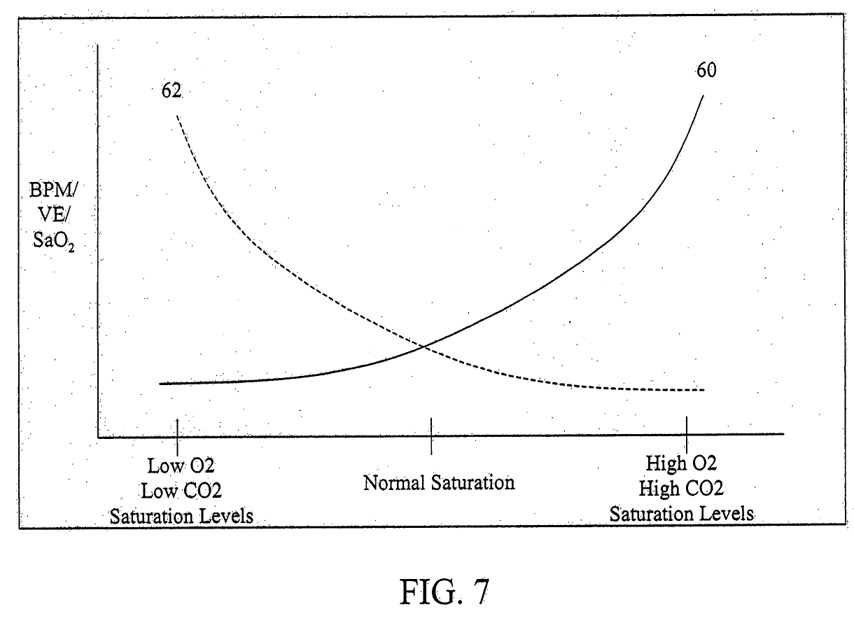

Reference is now made to FIG. 7, which is a graphical illustration of expected results in normal and RDB individuals exposed to low percentages of oxygen and carbon dioxide and exposed to high amounts of oxygen and carbon dioxide. The x-axis shows different levels of gas saturations. The y-axis shows VE, breathing rate, or SaO₂. The solid line 60 represents normal individuals, and the dashed line 62 represents RDB individuals.

If provided with low percentages of oxygen and carbon dioxide, normal individuals would be expected to react according to the more sensitive central chemoreceptors, and respond to the decrease in CO₂ by decreasing breathing rate and, consequently, decreasing the amount of VE or SaO₂. However, RDB individuals' would not react to the same extent to the decrease in CO₂ and would consequently react more to the low oxygen levels, increasing their breathing rate and amount of VE or SaO₂. Conversely, if provided with high percentages of oxygen and carbon dioxide, normal individuals would be expected to react according to the more sensitive central chemoreceptors, and respond to the increase in CO₂ by increasing breathing rate and, consequently, increasing the amount of VE or SaO₂. However, RDB individuals would not react with the same intensity to the increase in CO₂ and would consequently react more to the higher oxygen levels, decreasing their breathing rate and VE and amount of SaO₂. Providing a combination of high oxygen and low carbon dioxide or vice versa would not allow for a differential diagnosis as described above, since both normal and RDB individuals would be expected to decrease breathing rate with low percentages of oxygen and high percentages of carbon dioxide, and both normal and RDB individuals would be expected to increase breathing rate with high percentages of oxygen and low percentages of carbon dioxide.

An additional method for testing includes measurement of exhaled CO₂ during exercise, wherein the breathing rate of normal subjects would be expected to increase more than the breathing rate of RDB individuals. Normal exhaled PCO₂ is approximately 32 mmHg. A greater than 5% deviation from this number would indicate an abnormality.

It is appreciated that certain features of the invention, which are, for clarity, described in the context Of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference: In addition; citation or identification of any reference in this application shall not be construed as an admission that such-reference is available as prior art to the present invention.

1. A method of diagnosing RCC abnormalities comprising:
determining in a subject a physiological response to predetermined carbon dioxide levels so as to obtain a first value;
determining in a subject a physiological response to predetermined oxygen levels so as to obtain a second value;
combining said first and second values for a differential comparison; and
diagnosing an RCC disorder in the subject based on said comparison.

2. The method of claim 1, wherein said physiological response is arterial oxygen saturation.

3. The method of claim 1, wherein said physiological response is respiratory volume.

4. The method of claim 1, wherein said physiological response is breathing rate.

5. The method of claim 2, wherein said determining is done using a pulse oximeter.

6. The method of claim 3, wherein said determining is done using an air flow sensor.

7. The method of claim 4, wherein said determining is done using a pressure sensor.

8. The method of claim 1, wherein said physiological response is selected from the group consisting of: heart rate, pulse and blood flow.

9. The method of claim 1, wherein said predetermined carbon dioxide and oxygen levels includes normal air.

10. The method of claim 1, wherein said predetermined oxygen levels includes 5-12% oxygen.

11. The method of claim 1, wherein said predetermined oxygen levels includes 30-100% oxygen.

12. The method of claim 1, wherein said predetermined carbon dioxide levels includes less than 0.02% carbon dioxide.

13. The method of claim 1, wherein said predetermined carbon dioxide levels includes 1-10% carbon dioxide.

14. The method of claim 1, wherein said determining is done over 10-60 seconds.

15. The method of claim 1, wherein said determining is done over 1-20 minutes.

16. The method of claim 1, wherein said differential comparison is made between normal and RCC disorder subjects.

17. The method of claim 1, wherein said RCC disorder is sleep apnea.

18. The method of claim 1, wherein said RCC disorder is sudden infant death syndrome.

19. A method of diagnosing RCC abnormalities comprising:
determining in a subject at least one physiological response to predetermined carbon dioxide and oxygen levels; and
diagnosing a sleep disorder in the subject based on said determining.

20. The method of claim 19, wherein said physiological response is arterial oxygen saturation.

21. The method of claim 19, wherein said physiological response is respiratory volume.

22. The method of claim 19, wherein said physiological response is breathing rate.

23. The method of claim 20, wherein said determining is done using a pulse oximeter.

24. The method of claim 21, wherein said determining is done using an air flow sensor.

25. The method of claim 22, wherein said determining is done using a pressure sensor.

26. The method of claim 19, wherein said physiological response is selected from the group consisting of: heart rate, pulse and blood flow.

27. The method of claim 19, wherein said predetermined carbon dioxide and oxygen levels includes normal air.

28. The method of claim 19, wherein said predetermined carbon dioxide and oxygen levels includes 5-12% oxygen.

29. The method of claim 19, wherein said predetermined carbon dioxide and oxygen levels includes 30-100% oxygen.

30. The method of claim 19, wherein said predetermined carbon dioxide and oxygen levels includes less than 0.02% carbon dioxide.

31. The method of claim 19, wherein said predetermined carbon dioxide and oxygen levels includes 1-10% carbon dioxide.

32. The method of claim 19, wherein said determining is done over 10-60 seconds.

33. The method of claim 19, wherein said determining is done over 1-20 minutes.

34. The method of claim 19, wherein said diagnosing is done using a differential between normal and sleep disorder subjects.

35. The method of claim 19, wherein said sleep disorder is sleep apnea.

36. The method of claim 19, wherein said sleep disorder is sudden infant death syndrome.

37. A method for providing a diagnostic measure of a RCC disorder, the method comprising:
providing to a subject a predetermined ratio of oxygen and carbon dioxide;
measuring a breathing parameter of the subject in response to said provided combination;
comparing said breathing parameter to a baseline parameter; and
calculating an index based on said comparison, said index serving as said diagnostic measure.

38. The method of claim 37, further comprising reporting said index.

39. The method of claim 38, wherein said reporting is done via wireless communication.

40. The method of claim 38, wherein said reporting is done to a health care; provider.

41. The method of claim 37, wherein said predetermined ratio includes normal air.

42. The method of claim 37, wherein said predetermined ratio includes 5-12% oxygen and less than 0.02% carbon dioxide.

43. The method of claim 37, wherein said predetermined ratio includes 30-100% oxygen and 1-10% carbon dioxide.

44. The method of claim 37, wherein said predetermined ratio includes 5-12% oxygen and 1-10% carbon dioxide.

45. The method of claim 37, wherein said predetermined ratio includes 30-100% oxygen and less than 0.02% carbon dioxide.

46. The method of claim 37, wherein said predetermined ratio includes a variable amount of oxygen and carbon dioxide.

47. The method of claim 37, wherein said measuring is performed at rest.

48. The method of claim 37, wherein said measuring is performed during or after physical activity.

49. The method of claim 37, wherein said breathing parameter includes arterial saturated oxygen.

50. The method of claim 37, wherein said breathing parameter includes breathing rate.

51. A system for diagnosing a RCC disorder, the system comprising:
a device for determining in a subject at least one physiological response to predetermined combined carbon dioxide and oxygen levels; and
a processor for diagnosing the RCC disorder based on said physiological response.

52. The system of claim 51, said device comprising:
a gas mixer having therein a gas: mixture of a predetermined ratio of oxygen and carbon dioxide;
a gas introducer connected to said breathing gas mixer for introducing said gas mixture to a user;
a sensor connected to said user and said processor for measurement of at least one breath reaction parameter.

53. The system of claim 51, wherein said predetermined combined ratio includes normal air.

54. The system of claim 51, wherein said predetermined combined ratio includes 5-12% oxygen and less than 0.02% carbon dioxide.

55. The system of claim 51, wherein said predetermined combined ratio includes 30-100% oxygen and 1-10% carbon dioxide.

56. The system of claim 51, wherein said predetermined combined ratio includes 5-12% oxygen and 1-10% carbon dioxide.

57. The system of claim 51, wherein said predetermined combined ratio includes 30-100% oxygen and less than 0.02% carbon dioxide.

58. The system of claim 51, wherein said predetermined combined ratio includes a variable amount of oxygen and carbon dioxide.

59. The system of claim 51, wherein said processor comprises a gas regulator in communication with said breathing gas mixer, an input data collector in communication with said sensor, and an index calculator in communication with said input data collector.

60. The system of claim 51, further comprising a display in communication with said processor for display of said calculated index.

61. The system of claim 52, wherein said gas introducer is a mask.

62. The system of claim 52, wherein said gas introducer is a breathing chamber.

63. A diagnostic system, comprising:
a breathing gas mixer having therein a gas mixture which comprises a predetermined ratio of oxygen and carbon dioxide;
a breathing gas introducer connected to said breathing gas mixer for introducing said gas mixture to a user;
a sensor connected to said user for measurement of at least one breath reaction parameter;
a processor, wherein said processor comprises a gas regulator in communication with said, breathing gas mixer, an input data collector in communication with said sensor, and an index calculator in communication with said input data collector; and
a display in communication with said output data transmitter for display of said calculated index.

64. The diagnostic system of claim 63, wherein said predetermined ratio includes 5-12% oxygen and less than 0.02% carbon dioxide.

65. The system of claim 63, wherein said predetermined ratio includes 30-100% oxygen and 1-10% carbon dioxide.

66. The system of claim 63, wherein said predetermined ratio includes 5-12% oxygen and 1-10% carbon dioxide.

67. The system of claim 63, wherein said predetermined ratio includes 30-100% oxygen and less than 0.02% carbon dioxide.

68. The system of claim 63, wherein said predetermined ratio includes a variable amount of oxygen and carbon dioxide.

69. The diagnostic system of claim 63, wherein said breathing gas introducer is a mask.

70. The diagnostic system of claim 63, wherein said breathing gas introducer is a chamber.

71. The diagnostic system of claim 63, wherein said sensor is a pressure sensor.

72. The diagnostic system of claim 63, wherein said breath reaction parameter is oxygen/hemoglobin saturation.

73. The diagnostic system of claim 63, wherein said breath reaction parameter is breathing rate.

74. The diagnostic system of claim 63, wherein said breath reaction parameter is respiratory volume.

75. The system of claim 63, wherein said breath reaction parameter is, selected from the group consisting of: sympathetic nerve activity, heart rate, pulse and blood flow.

76. The system of claim 63, wherein said processor is configured- to transmit over a wireless connection.

77. The system of claim 63, wherein said display is a results report.

78. The system of claim 63, wherein said display is graph.

79. A system for providing a diagnostic measure of sleep apnea, comprising:
a breathing gas, combination provider; and
a breathing parameter measurer for measuring at least one breathing parameter of a user in response to breathing said breathing gas combination.

80. The system of claim 79, further comprising:
a comparator for comparing said measurement to a predetermined baseline parameter; and
an evaluator for providing a diagnosis based on said comparison.

81. The device of claim 79, wherein said gas combination provider comprises a preparation chamber and an introducer.

82. The device of claim 81, wherein said preparation chamber has therein a gas mixture of a predetermined ratio of oxygen and carbon dioxide.

83. The device of claim 82, wherein said predetermined ratio includes normal air ratios.

84. The device of claim 82, wherein said predetermined ratio includes 5-12% oxygen.

85. The device of claim 82, wherein said predetermined ratio includes 30-100% oxygen.

86. The device of claim 82, wherein said predetermined ratio includes less than 0.02% carbon dioxide.

87. The device of claim 82, wherein-said predetermined ratio includes 10% carbon dioxide.

88. The device of claim 81, wherein said introducer is a mask.

89. The device of claim 81, wherein said introducer is a breathing chamber.

90. The device of claim 79, wherein said breath parameter is artenral oxygen saturation.

91. The device of claim 79 wherein said breath parameter is breathing rate.

92. The device of claim 79, wherein said breath parameter is respiratory volume.
